SEQUENCE LISTING (2 sequences)

SEQ ID NO: 1 - protein, 1273 aa, severe acute respiratory syndrome coronavirus 2
  “SARS-CoV-2 spike protein”; SARS-CoV-2 spike protein
  SARS-CoV-2 spike protein
  SARS-CoV-2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 223 aa, severe acute respiratory syndrome coronavirus 2
  “RBD domain of the SARS-CoV-2 spike protein”; RBD domain of the SARS-CoV-2 spike protein
  RBD domain of the SARS-CoV-2 spike protein
  RBD domain of the SARS-CoV-2 spike protein
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

(19) United States
(12) Patent Application Publication
(10) Pub. No.: US 2024/0294612 A1
(43) Pub. Date: Sep. 5, 2024

(54) COMPOSITION OF PIG POLYCLONAL ANTIBODY FOR ITS USE TO TREAT AND/OR PREVENT ANTIBODY-DEPENDENT MACROPHAGE PRO-INFLAMMATORY CYTOKINE RELEASE IN A PASSIVE ANTI-INFECTIOUS IMMUNOTHERAPY

(71) Applicant: XENOTHERA, Nantes (FR)

(72) Inventors: Odile DUVAUX, Nantes (FR); Bernard VANHOVE, Rezé (FR); Carine CIRON, Le Palais (FR)

(21) Appl. No.: 18/268,720

(22) PCT Filed: Dec. 22, 2021
(86) PCT No.: PCT/EP2021/087205

§ 371 Date: Jun. 21, 2023

Related U.S. Application Data
(60) Provisional application No. 63/129,964, filed on Dec. 23, 2020.

(30) Foreign Application Priority Data
Dec. 23, 2020 (EP) 20306688.1

(51) Int. Cl. C07K 16/10; A61K 39/00

(52) U.S. Cl. CPC C07K 16/1003; A61K 2039/505; C07K 2317/20; C07K 2317/33; C07K 2317/76; C07K 2317/92

(57) ABSTRACT

The present invention relates to a pig polyclonal antibody composition for its use in preventing or treating a macrophage-dependent inflammation's disease induced by at least one virus wherein said inflammation is characterized by a cytokine storm in a human subject to, or susceptible to be subjected to, the disease, wherein the polyclonal antibodies of the composition are directed against the said at least one virus, or against at least one molecule derived from the said virus, the composition comprising a pharmaceutically acceptable excipient.

### FIELD OF THE INVENTION

The present invention relates to the field of treatment and/or prevention of infectious diseases. In particular, the present invention relates to the field of passive anti-infectious immunotherapies and more particularly to the use of polyclonal antibodies in the prevention or treatment of the antibody-dependent macrophage pro-inflammatory cytokine release observed during such diseases or occurring after treatment or after vaccination against an infectious agent.

### DESCRIPTION OF RELATED ART

For the past few months, almost one year following the first human Sars-Cov-2 infection and subsequent pandemia, many scientists have fought, and are still fighting, to discover a vaccine or therapeutic solutions against this disease. Many efforts are deployed to understand how this virus may be so infectious and why some patients develop deleterious symptoms not directly due to the virus.

Indeed, it was determined that 20% of the COVID-19 patients showed a severe disease outcome worsening around 1 to 2 weeks after the onset of symptoms. Scientific people agree that it is not a direct effect of the virus infection but that it instead seems to be caused by an over-activation of the immune system, particularly because it coincides with the activation of adaptive immunity. This excessive immune response is frequently described as a ‘cytokine storm’, characterized by high levels of pro-inflammatory cytokines. Some cytokines and chemokines are particularly elevated, such as IL-6, IL-8, and TNF. Hoepel et al. lately described (Jul. 13, 2020-doi: https://doi.org/10.1101/2020.07.13.190140) that in a model where Spike-IgG immune complexes were generated by incubating SARS-COV-2 Spike-coated wells with diluted serum from severely ill COVID-19 patients positive for anti-SARS-COV-2 IgG, stimulation with Spike protein alone did not induce cytokine production, while Spike-IgG immune complexes elicited small amounts of IL-1B, IL-6, and TNF, but very high IL-8 production by human macrophages. They concluded that anti-Spike IgG from serum of severely ill COVID-19 patients strongly amplify pro-inflammatory responses by human macrophages and can contribute to subsequent endothelial barrier disruption and thrombosis.

The cytokine storm is a phenomenon not limited to human SARS-COV-2 infections and has previously been observed in the macrophage activation syndrome (MAS) also known as the Hemophagocytic syndrome. This syndrome results from an inappropriate stimulation of macrophages in bone marrow and lymphoid organs, leading to phagocytosis of blood cells and production of high amounts of pro-inflammatory cytokines. This life-threatening disease combines non-specific clinical signs (fever, cachexia, hepatomegaly, enlargement of spleen and lymph nodes) as well as typical laboratory findings (bi- or pancytopenia, abnormal hepatic tests, hypofibrinemia, elevation of serum LDH, ferritin and triglyceride levels). MAS or secondary Hemophagocytic lymphohistiocytosis (HLH) is a poorly recognized syndrome characterized by a fulminant cytokine storm, multiple organ dysfunction, and a high mortality rate. MAS can occur during an autoimmune disease, a tumor, and even during an infectious disease. Viral infections have especially been linked to this syndrome in adults. An inappropriate immune stimulation and a self-perpetuating excessive inflammatory response are key facts within the pathogenesis of MAS, and the over-activation of tissue macrophages for the release of a storm of cytokines is a dominant feature observed both in MAS and severe COVID-19 patients (scc https://doi.org/10.1016/j.lfs.2020.117905).

In general, virus-specific antibodies are considered as being advantageous in fighting the diseases caused by said pathogen and to play an important role in the control of the corresponding infection in a number of ways. However, in some instances, the presence of specific antibodies can be beneficial to the pathogen, and in particular if the said pathogen is a virus. This unwanted effect is known as antibody-dependent enhancement (ADE).

For the inventors, pig was chosen for several reasons.
1) Safety issues related to the use of that species have been studied thoroughly owing to numerous medical indications related to the use of biomolecules and biomaterials from porcine origin including insulin, heparin, but also heart valves, skin, tendons, and functional cells such as pancreatic islets constituting an artificial bio pancreas for which clinical trials are already underway. A pig IgG preparation is also in use in the clinic for decades to fight against Severe Aplastic Anemia (Wuhan Biologics, Peking Union Medical College Hospital, Pekin, China).
2) Immune sera presenting higher titers can be obtained in DKO animals compared to wild-type animals, a phenomenon possibly due to the lower activity of the Siglec immune checkpoints that are normally interacting with sialic acids.
3) Use of pigs will permit to reduce the number of animals needed for the production of polyclonals, due to their higher weight compared to rabbits.
4) Porcine immunoglobulins of the IgG class naturally present a better complement-mediated cytotoxic activity.

In some interesting and unexpected way, the inventors published lately the inability of pig Glyco-Humanized Polyclonal Antibodies (GH-pAbs) to bind human FcG-R (CD12, CD32, CD64), an interesting feature that might protect against Antibody-Dependent Enhancement (ADE), a process by which several viruses (among which dengue, Zika, SARS-COV) have an increased capacity to enter into cells expressing FcG receptors in the presence of endogenous or exogenous antibodies capable of binding human FcG receptor Vanhove et al., July 2020 BioRxiv (doi: https://doi.org/10.1101/2020.07.25.217158). The mechanisms of action are viral particle opsonization and FcGR-dependent cell entry.

Inventors published in correlation with the inability of the pig antibody to bind human Fc Receptor that the same pig polyclonal antibodies are unable to induce antibody dependent cell cytotoxicity (ADCC) suggesting that pig polyclonal antibodies would not elicit antibody-dependent enhancement (ADE) in opposite to the other immunoglobulins from different species such as rabbit, bovine, goat. Vanhove et al., July 2020 BioRxiv (doi: https://doi.org/10.1101/2020.07.25.217158).

More recently, this phenomenon has been reported in a huge number of infections by viruses in vitro and in vivo, in both the public health and veterinarian fields. ADE has thus become a consequent drawback of current vaccinal and passive immunization strategies.

WO2016059161 reports antibodies directed against non-human biological pathogens, which are devoid of an antigenic determinant selected in a group comprising (i) N-glycolylneuraminic acid (Neu5Gc) and/or (ii) a-1.3-galactose, which are characterized by their reduced immunogenicity. However, this document is silent on, specifically, ADE

Scientifics are trying to develop novel treatments, and in particular antibodies, with reduced or no risk of ADE, by using molecular constructs producing recombinant antibody or antigen binding thereof.

Pig polyclonal antibodies are naturally not capable of binding human Fc receptors and those antibody are not able to induce ADCC in vitro or in vivo in human models.

There is a need in the art for treatment directed against novel virus for which ADE may be detrimental. This is particularly the case for viruses belonging to the Coronaviridae family, such as SARS-COV-2. The ADE mechanism is not the only one involved in the worsening of the human infection, but cytokine storm is an important event that must be controlled or avoided.

There is also a need for novel treatments, and in particular antibodies, reducing or inhibiting macrophage-dependent inflammatory cytokine storm during infections, in particular during viral infections, more particularly during infections by viruses from the Coronaviridae family, and even more particularly during infections by SARS-Cov-2.

The invention has for purpose to meet the above-mentioned needs.

### SUMMARY OF THE INVENTION

According to a first of its aspects, the invention relates to a pig polyclonal antibody composition for its use in preventing or treating a macrophage-dependent inflammation's disease induced by at least one virus wherein said inflammation is characterized by a cytokine storm in a human subject to, or susceptible to be subjected to, the disease,
wherein the polyclonal antibodies of the composition are directed against the said at least one virus, or against at least one molecule derived from the said virus,
the composition comprising a pharmaceutically acceptable excipient.

The said antibody can be devoid of the two antigenic determinants (i)N-glycolylneuraminic acid (Neu5Gc) and (ii) a-1,3-galactose. The virus towards a human subject can be selected from the group consisting of Coronaviridae family, such as SARS-CoV-2; Dengue virus; Zika virus; Ebola virus; human immunodeficiency virus (HIV); Influenza B virus, hepatitis C virus, Japanese encephalitis virus, Aleutian mink disease parvovirus (AMDV), Human enterovirus 71 (EV71), Ross River virus, Hantavirus, yellow fever virus and a combination thereof.

The said polyclonal antibodies of the composition according to the invention can be Immunoglobulin G.

The disease induced by at least one virus can be an infection. In particular, said infection can be selected from the group consisting of:
Coronaviridae-related infections, such as SARS, MERS or COVID-19;
Dengue fever, in particular dengue hemorrhagic fever or dengue shock syndrome;
Zika fever disease;
Influenza, in particular influenza due to the Influenza B virus;
Ebola virus disease;
Acquired immunodeficiency syndrome (AIDS);
Encephalitis, in particular Japanese encephalitis;
Aleutian disease;
hepatitis, in particular hepatitis C;
neurological diseases, and in particular neurological diseases caused by Human enterovirus 71 (EV71);
hand, foot and mouth disease (HFMD);
Ross River fever;
diseases caused by Hantavirus, such as hantavirus hemorrhagic fever with renal syndrome (HFRS) or hantavirus cardiopulmonary syndrome (HCPS); and
yellow fever.

In a particular embodiment, the disease induced by at least one virus is an infection by a RNA virus.

The disease can in particular be an infection by a virus belonging to the Coronaviridae family, in particular selected from the group consisting of: SARS-COV, SARSr-CoV WIV1, SARS-COV HKU3, SARS-COV RP3, SARS-COV-2, and their mutants.

In a particular embodiment, the disease can be Hemophagocytic lymphohistiocytosis (HLH).

The cytokine storm of the macrophage-dependent's inflammation can in particular be characterized by an uncontrolled and excessive release of at least one of the cytokines selected from the group consisting of: Interleukin 8 (IL-8), granulocyte colony-stimulating factor (G-CSF), Interleukin 6 (IL6), TNFalpha, Interleukin 1β (IL1β), MCP-1, MIP1 alpha and beta CCL-3, CCL-4, CXCL-10 and (https://doi.org/10.1016/j.cytogfr.2020.06.001).

The polyclonal antibodies of the composition can in particular have a binding activity by ELISA to the virus or to the at least one molecule derived from the said virus (EC50) between 0.05 μg/mL and 6 μg/mL. The at least one molecule derived from the virus can in particular be the spike protein of SARS-COV-2 or a fragment thereof, and is in particular selected from the group consisting of the amino acid sequences as set forth in sequences SEQ ID NO: 1 and SEQ ID NO: 2, and is more particularly the amino acid sequence as set forth in sequence SEQ ID NO: 2.

The polyclonal antibodies of the composition can in particular have a neutralization activity by ELISA to the virus or to the at least one molecule derived from the said virus (IC50) between 0.10 μg/mL and 11 μg/mL. The at least one molecule derived from the virus can in particular be the spike protein of SARS-COV-2 or a fragment thereof, and is in particular selected from the group consisting of the amino acid sequences as set forth in sequences SEQ ID NO: 1 and SEQ ID NO: 2, and is more particularly the amino acid sequence as set forth in sequence SEQ ID NO: 2.

The present invention also relates to the pig polyclonal antibody composition for its use in preventing or treating the occurrence of antibody-dependent macrophage skewing in a human subject to, or susceptible to be subjected to, a disease induced by a virus, in particular a virus as previously defined, wherein:
wherein the polyclonal antibodies of the composition are directed against the said at least one virus, or against at least one molecule derived from the said virus,
the composition comprising a pharmaceutically acceptable excipient.

The present invention also relates to the composition for its use in preventing or treating the occurrence of antibody-dependent macrophage skewing in a human subject to, or susceptible to be subjected to, a disease induced by at least one virus, in particular a virus as defined above, wherein:
wherein the polyclonal antibodies of the composition are directed against the said at least one virus, or against at least one molecule derived from the said virus,
the composition comprising a pharmaceutically acceptable excipient.

Indeed, the antibodies according to the invention are particularly convenient for the treatment and/or prevention of infections caused by a virus belonging to the Coronaviridae family. Without wishing to be bound by theory, the inventors are of the opinion that the following characteristics are particularly efficient for treating and preventing such viruses: pig immunoglobulins were shown to neither bind to human CD16, nor to CD32 or CD64, whereas antibodies directed against the spike protein of SARS-COV-2 have been shown in other studies to induce pulmonary inflammation, by recruiting macrophages in lungs through their Fc part, leading to the production of chemokines such as MCP-1 and IL-8. This phenomenon has been proposed to lead to the recruitment of other inflammatory cells, which tend to hinder wound-healing. Accordingly, the polyclonal antibodies of the invention are able to combine viral neutralization with no, or little, induction of cytokine storm dependent of the macrophage.

A composition according to the invention can in particular reduce the uncontrolled and excessive release of at least one cytokine, in particular at least one cytokine selected from the group consisting of Interleukin 8 (IL-8) and granulocyte colony-stimulating factor (G-CSF), in the above-mentioned human subject by at least 10%, 20%, 30%, 40%, 50%, 60%, 70%, 80%, 90% or 95%, and more particularly by 50% to 95%, as compared to the said uncontrolled and excessive release of the said at least one cytokine in the human subject not administered with the composition.

### BRIEF DESCRIPTION OF THE FIGURES

FIG. 1: illustrates the binding activity of the pig DKO anti-RBD polyclonal antibody (pAb) generated after pig vaccination compare to irrelevant pig DKO pAb or blank (Blk) by a binding ELISA.

FIG. 2: illustrated the Inhibition/Neutralization of the Spike-ACE interaction in vitro by ELISA
A/represents the percentage of inhibition of spike-ACE interaction by purified IgG (DKO anti-RBD pAb) from 5 different immunized animals.
B/represents SARS-COV-2 spike Protein interaction with ACE2 generating a signal in the ELISA which can be inhibited by antibodies neutralizing this interaction. 100% inhibition represents complete abrogation of the interaction. The concentration of DKO anti-RBD pAb (IgG purified fraction) by protein A capture chromatography is reported in the x-axis (μg/ml) while the % of inhibition is reported in the y-axis. Dots and squares: two different preparations from sera; triangles: IgG from non-immunized pigs.

FIG. 3: represents the correlation curve between binding (y-axis) and neutralization (x-axis) on IgG from five different animals: data obtained after A/course 1 of immunization; B/course 2 of immunization and C/course 3 of immunization.

FIG. 4: represents the phagocytosis activity of macrophages in presence of pig DKO pAb: the percentage of the macrophage phagocytosis activity was determined at 4° C. and 37° C. and DKO pAb was compared to an IgG control.

FIG. 5: represents the in vitro model of macrophage activation by virus: IL-8 and G-CSF cytokine secretion study by ELISA on U937 (A) or THP1 (B) cell lines pre activated with PMA and in presence of lentivirus expressing S1 and S2 domains of the spike SARS-COV-2 protein. The pig DKO anti-RBD pAb was compared to a rabbit polyclonal antibody (pAb Rb). Statistical analysis: one way anova-post-hoc test Fischer, ** p<0,01.

FIG. 6: illustrates the in vitro model of macrophage activation by virus: IL-8 cytokine secretion study by ELISA on U937 cell line pre activated with PMA and in presence of lentivirus expressing S1 and S2 domains of the spike SARS-COV-2 protein. The pig DKO anti-RBD pAb was compared to polyclonal IgG from human infected by SARS-COV-2 (human pAb). Statistical analysis: one way anova-post-hoc test Fischer, ** p<0,01.

### DETAILED DESCRIPTION OF THE INVENTION

### 1. Definitions

Several definitions are set forth below. Such definitions are meant to encompass grammatical equivalents.

As used herein, the term “comprising” encompasses “consisting of”.

By “pharmaceutically acceptable excipient” is meant any solvent, dispersion medium, charge etc, which does not produce a side reaction, for example allergic, in humans or animals. The excipient is selected according to the chosen dosage form, method and route of administration. Suitable excipients, as well as pharmaceutical formulation requirements, are described in “Remington: The Science & Practice of Pharmacy”, which is a reference work in the field.

The term “antibody” is used herein in the broadest sense. “Antibody” refers to any polypeptide which at least comprises (i) a Fc region and (ii) a binding polypeptide domain derived from a variable region of an immunoglobulin. Antibodies thus include, but are not limited to, full-length immunoglobulins, antibodies, antibody conjugates and fragments of each respectively. The terms “antibody” and “immunoglobulin” may be used interchangeably herein.

The term “antibody” encompasses a polypeptide as above-mentioned, and in particular an antibody from a pig, which further comprises at least one sugar moiety distinct from the antigenic determinant selected in a group comprising (i)N-glycolylneuraminic acid (Neu5Gc) and/or (ii) a-1,3-galactose.

By “polyclonal antibodies or polyclonal antibody” as used herein is meant a mixture of antibodies recognizing different epitopes of a given antigen. Polyclonal antibodies encompass those which are contained in, or alternatively which are derived from, body fluids, especially serum or plasma from a non-human mammal organism, in particular a pig.

The choice of polyclonal antibodies over monoclonal antibodies for selected antibody therapeutics today makes sense because polyclonal antibodies (pAbs) are antibodies that are secreted by highly diverse B cell and plasmacytes lineages within the body (whereas monoclonal antibodies come from a single cell lineage). Thus, polyclonal hyper immune antibodies are a collection of highly diverse immunoglobulin molecules, of various classes and isotypes that react against a specific antigen, but each identifying a different epitope. As example, rabbit polyclonal IgG raised against human T cells interact against at least 50 clusters of differentiation (CDs) (Popov et al., Transplantation, 2012). On the contrary, associations of monoclonal antibodies are limited to few molecules and cannot mimic the polyclonal diversity of polyclonal antibodies.

In addition, polyclonal antibodies act through a variety of mechanisms (notably complement—and cell-dependent cytotoxicity (CDC and ADCC respectively), neutralization, opsonization, etc.) that only a variety of molecular target and Ig classes and isotypes can offer and which cannot be replicated by a monoclonal antibody, or even by associations of monoclonal antibodies.

Hence, usage of passive serotherapy using sera or purified polyclonal immunoglobulins from animals (rabbit, horse, goat) has been a first major advance in treating or preventing the dissemination of severe infectious diseases, such as Plague or Diphteria.

Within the general meaning of the present invention, a polyclonal antibody in a composition according to the invention is directed against at least one virus, or against at least one molecule derived from said virus, for a human organism.

In the case of human immunoglobulins, light chains are classified as kappa and lambda light chains. Heavy chains are classified as mu, delta, gamma, alpha, or epsilon, and define the antibody's isotype as IgM, IgD, IgG, IgA, and IgE, respectively.

By “IgG” as used herein is meant a polypeptide belonging to the class of antibodies that are substantially encoded by a recognized immunoglobulin gamma gene. In humans, IgG comprises the subclasses or isotypes IgG1, IgG2, IgG3, and IgG4. In mice, IgG comprises IgG1, IgG2a, IgG2b, IgG3. In pigs, immunoglobulins comprise the classes or isotypes IgM, IgD, IgG, IgE and IgA antibodies and the IgG isotype comprise 11 subclasses (Butler et al., Developmental and Comparative Immunology 30 (2006) 199-221; Butler et al., Developmental and Comparative Immunology 33 (2009) 321-333). Full-length IgGs consist of two identical pairs of two immunoglobulin chains, each pair having one light and one heavy chain, each light chain comprising immunoglobulin domains VL and CL, and each heavy chain comprising immunoglobulin domains VH, Cγ1 (also called CH1), Cγ2 (also called CH2), and Cγ3 (also called CH3).

By “antigenic determinant” (or epitope), as applied herein to pig antibodies, as used herein is meant a structural component of an antigenic molecule, which includes an antigenic protein and an antigenic carbohydrate, responsible for its specific interaction with antibody molecules elicited by the same or related antigen. By extension, the term “antigenic determinant”, as applied herein to pig antibodies is also used collectively herein for an antigenic molecule comprising a plurality of epitopes, including conformational motives in which the sugar moiety is needed but represent only part of the epitope, susceptible to be recognized by antibody molecules elicited by the same or related antigen. Illustratively, the antigenic molecule N-glycolylneuraminic acid (Neu5Gc) may be called herein an “antigenic determinant”, although the said antigenic molecule may exhibit more than one epitope recognized by antibodies elicited with Neu5Gc or with Neu5Gc containing molecules.

“T cells” or “T lymphocytes” belong to a group of white blood cells known as lymphocytes and play a central role in cell-mediated immunity. They can be distinguished from other lymphocytes, such as B cells and natural killer cells (NK cells), by the presence of a T-cell receptor (TCR) on the cell surface. They are called T cells because they mature in the thymus.

By “conventional polyclonal antibodies”, as used herein is meant polyclonal antibodies, and in particular pig polyclonal antibodies, that are not devoid of the antigenic determinants N-glycolylneuraminic acid (Neu5Gc) and a-1,3-galactose. In this regard, it may be notably cited the products commercialized under the name Thymoglobulin, Grafalon, Atgam or p-ALG®.

The term “pathogen” is herein used to mean a virus that causes disease or infection in its human host.

As used herein the term “virus” may encompass all viruses belonging to the Baltimore classification.

For reference, the content of the “Baltimore classification” which is reported herein further references to the virus taxonomy as set forth in the database of the International Committee of Taxonomy of Viruses (ICTV) as available online on Mar. 20, 2020 ratification 2019 & MSL at (Email February #34) https://talk.ictvonline.org/taxonomy/. This taxonomy is incorporated herein in its entirety.

Accordingly, this classification clusters viruses into families (or “groups”) depending on their type of genome. The present virus classification, as in 2018, comprises seven different groups:
Group I: double-stranded DNA viruses (dsDNA);
Group II: single-stranded DNA viruses (ssDNA);
Group III: double-stranded RNA viruses (dsRNA);
Group IV: (+)strand or sense RNA viruses ((+)ssRNA);
Group V: (−)strand or antisense RNA viruses ((−)ssRNA);
Group VI: single-stranded RNA viruses having DNA intermediates (ssRNA-RT);
Group VII: double-stranded DNA viruses having RNA intermediates (dsDNA-RT).

As used herein the term “RNA virus” may encompass all viruses belonging to the Group IV and V of the Baltimore classification.

As used herein the term “retrovirus” may encompass all viruses belonging to the Group VI of the Baltimore classification.

As used herein, the term “Coronaviridae” refers to the corresponding family of RNA viruses belonging to the group IV of the Baltimore classification, which is iself part of the Coronidovirineae suborder and of the Nidovirales Order. The Coronaviridae family includes both the Letovirinae and Orthocoronavirinae subfamilies.

As used herein, the term “Letovirinae” refers to the corresponding family of the Baltimore classification, which includes the Alphaletovirus genus, the Milecovirus subgenus, which includes (in a non-exhaustive manner) the Microhyla letovirus 1 species.

As used herein, the term “Orthocoronavirinae” refers to the corresponding family of the Baltimore classification, which includes the Alphacoronavirus, Betacoronavirus, Deltacoronavirus, and Gammacoronavirus genus.

As used herein, the term “Alphacoronavirus” refers to the corresponding family of the Baltimore classification, which includes the Colacovirus, Decacovirus, Duvinacovirus, Luchacovirus, Minacovirus, Minunacovirus, Myotacovirus, Myctacovirus, Pedacovirus, Rhinacovirus, Setracovirus, and Tegacovirus subgenus. In a non-exhaustive manner, this includes the following species: Bat coronavirus CDPHE15, Bat coronavirus HKU10, Rhinolophus ferrumequinum alphacoronavirus HuB-2013, Human coronavirus 229E, Lucheng Rn rat coronavirus, Ferret coronavirus, Mink coronavirus 1, Miniopterus bat coronavirus 1, Miniopterus bat coronavirus HKU8, Myotis ricketti alphacoronavirus Sax-2011, Nyctalus velutinus alphacoronavirus SC-2013, Porcine epidemic diarrhea virus, Scotophilus bat coronavirus 512, Rhinolophus bat coronavirus HKU2, Human coronavirus NL63, NL63-related bat coronavirus strain BtKYNL63-9b, Alphacoronavirus 1.

As used herein, the term “Betacoronavirus” refers to the corresponding family of the Baltimore classification, which includes the Embecovirus, Hibecovirus, Merbecovirus, Nobecovirus, and Sarbecovirus subgenus. In a non-exhaustive manner, this includes the following species: Betacoronavirus 1, China Rattus coronavirus HKU24, Human coronavirus HKU1, Murine coronavirus, Bat Hp-betacoronavirus Zhejiang2013, Hedgehog coronavirus 1, Middle East respiratory syndrome-related coronavirus, Pipistrellus bat coronavirus HKU5, Tylonycteris bat coronavirus HKU4, Hedgehog coronavirus 1, Middle East respiratory syndrome-related coronavirus, Pipistrellus bat coronavirus HKU5, Tylonycteris bat coronavirus HKU4, Rousettus bat coronavirus GCCDCl, Rousettus bat coronavirus HKU9, Severe acute respiratory syndrome-related coronavirus.

As used herein, the term “Severe acute respiratory syndrome-related coronavirus”, or SARS virus, includes, in a non-exhaustive manner, the SARS-COV, SARSr-CoV WIVI, SARSr-COV HKU3, SARSr-CoV RP3, and SARS-COV-2; including strains responsible for COVID-19 and their mutants.

As used herein, the term “Deltacoronavirus” refers to the corresponding family of the Baltimore classification, which includes the Andecovirus, Buldecovirus, Herdecovirus, and Moordecovirus subgenus. In a non-exhaustive manner, this includes the following species: Wigeon coronavirus HKU20, Bulbul coronavirus HKU11, Coronavirus HKU15, Munia coronavirus HKU13, White-eye coronavirus HKU16, Night heron coronavirus HKU19, Common moorhen coronavirus HKU21.

As used herein, the term “Gammacoronavirus” refers to the corresponding family of the Baltimore classification, which includes the Cegacovirus and Igacovirus subgenus. In a non-exhaustive manner, this includes the following species: Beluga whale coronavirus SWI and Avian coronavirus.

The present invention relates to a virus towards a human organism.

The terms “at least one molecule derived from the said virus” used herein encompass antigenic fractions derived from a virus (namely any molecules derived from said virus), which includes notably antigenic proteins or antigenic polysaccharides thereof.

The terms “antigenic fraction derived from a virus towards a human organism” as used herein refers broadly to any antigen from a virus to which a human organism can generate an immune response. This “molecule” or “antigen” as used herein refers to molecules that contain at least one antigenic determinant to which the immune response may be directed. The immune response may be cell mediated or humoral or both. The molecule derived from a virus may be protein in nature, carbohydrate in nature, lipid in nature, or nucleic acid in nature, or combinations of these biomolecules. The molecule derived from a virus may also include molecules such as polymers and the like.

The term “nucleic acid”, as used herein, encompasses ribonucleic acid (RNA) and deoxyribonucleic acid (DNA), which include nucleic acids selected from the group comprising or consisting of: single-stranded deoxyribonucleotide(s) (ssDNA); double-stranded deoxyribonucleotide(s) (dsDNA); single-stranded ribonucleotide(s) (ssRNA); double-stranded ribonucleotide(s) (dsRNA); single-stranded oligo-deoxyribonucleotide(s) (ssODNA); double-stranded oligo-deoxyribonucleotide(s) (dsODNA); single-stranded oligo-ribonucleotide(s) (ssORNA); double-stranded oligo-ribonucleotide(s) (dsORNA); RNA-DNA duplexes.

In a non-limitative manner, said nucleic acids may be in the form of messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers.

By a “therapeutically effective amount” of polyclonal antibodies used according to the invention is meant a sufficient amount of the antibody to prevent or treat the considered disease or disorder at a reasonable benefit/risk ratio applicable to any medical treatment. It will be understood, however, that the total daily usage of the antibodies present in compositions of the present invention will be decided by the attending physician within the scope of sound medical judgment. The specific therapeutically effective dose level for any particular patient will depend upon a variety of factors including the disorder being prevented; the activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

The terms “treatment”, “treating” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner, in the present case inflammation macrophage dependent CXCL10 and MIP1 alpha and beta.

The terms “prevent”, “preventing” and “prevention” refer to reducing the likelihood of acquiring a disease and/or one or all of its symptoms. In particular, regarding the prevention of the occurrence of MAS, it means reducing the likelihood that MAS appears in a human.

The term “wild type pig” comes herein in opposition with a genetically altered pig. For example, by “wild type pig”, is meant a pig which is not lacking at least one gene selected in a group comprising (i) a gene encoding a functional cytidine-5′-monophosphate N-acetyl neuraminic acid hydrolase (CMAH) and (ii) a gene encoding a functional α-(1,3)-galactosyltransferase.

As used herein, the term “biological sample” may encompass a biological fluid, such as a blood, a plasma, a serum, a saliva, an interstitial fluid, sperm or milk; a cell sample, such as a cell culture, a cell line, or a PBMC sample; a tissue biopsy, such as an oral tissue; a gastrointestinal tissue; a skin; an oral mucosa sample; or a pharyngeal, tracheal, bronchoalveolar sample.

### 2. Antibody Composition According to the Invention

As mentioned above, the present invention relates to a pig polyclonal antibody composition for its use in preventing or treating a macrophage-dependent inflammation's disease induced by at least one virus wherein said inflammation is characterized by a cytokine storm in a human subject to, or susceptible to be subjected to, the disease.

The polyclonal antibodies of a composition of the invention are characterized in that they are directed against the said at least one virus, or against at least one molecule derived from the said virus.

Moreover, a composition according to the invention comprises a pharmaceutically acceptable excipient.

The inventors have indeed published that polyclonal antibodies from a pig cannot bind to human FcγR and therefore are expected to not induce ADE or to inhibit ADE induced by autologous IgG antibodies. The antibodies of the composition reduce or even unexpectedly inhibit macrophage-dependent inflammatory cytokine storm during infections, in particular during viral infections while they are able to induce macrophage phagocytosis.

It has indeed been for example described that patients with severe form of Dengue disease produce antibodies with higher affinity for FcγRIIIa (Wang et al., Science 355, 395-398, 2017), that hemorrhage, coagulopathy and inflammation through FcγRIII ligation are observed during Dengue infections (Lien et al. Thrombosis and Haemostasis 113, 2015), that mast cell degranulation after FcγRIII ligation promotes vascular leakage during dengue infection (Syenina et al., eLife 2015;4:e05291) and that the inhibition of phagocytosis through FcγRIIb ligation reduces ADE and infection in dengue infected patients (Chan et al., PNAS 108, 12479-12484, 2011).

These polyclonal antibodies of a composition of the invention can thus advantageously be used in human passive immunotherapy.

A composition of the invention can also advantageously be used to reduce or even inhibit macrophage-dependent inflammatory cytokine storm during viral infections.

In particular, the cytokine storm of the inflammation is characterized by an uncontrolled and excessive release of at least one of the cytokines selected from the group consisting of: Interleukin 8 (IL-8), granulocyte colony-stimulating factor (G-CSF), Interleukin 6 (IL6), TNFalpha, Interleukin 1β (IL1β), MCP-1, CCL-3, CCL-4, CXCL-10 and MIP1alpha and beta.

In particular, polyclonal antibodies of a composition according to the invention advantageously have a binding activity (EC50) by ELISA to the virus or to the at least one molecule derived from the said virus between 0.05 and 6 μg/mL. The at least one molecule derived from the virus can in particular be the spike protein of SARS-COV-2 or a fragment thereof, and is in particular selected from the group consisting of the amino acid sequences as set forth in sequences SEQ ID NO: 1 and SEQ ID NO: 2, and is more particularly the amino acid sequence as set forth in sequence SEQ ID NO: 2.

In particular, polyclonal antibodies of a composition according to the invention advantageously have a neutralization activity (IC50) by ELISA to the virus or to the at least one molecule derived from the said virus the spike antigen of between 0.10 μg/mL and 11 μg/mL. The at least one molecule derived from the virus can in particular be the spike protein of SARS-COV-2 or a fragment thereof, and is in particular selected from the group consisting of the amino acid sequences as set forth in sequences SEQ ID NO: 1 and SEQ ID NO: 2, and is more particularly the amino acid sequence as set forth in sequence SEQ ID NO: 2.

Antibodies of a composition for its use according to the invention can be devoid of the two antigenic determinants N-glycolylneuraminic acid (Neu5Gc) and a-1.3-galactose.

Indeed, despite demonstrated efficacy, the injection in humans of immune immunoglobulins (IgG or IgM for instance) from animals may remain immunogenic and is responsible for the generation of immune complex related diseases (ICD) and severe unwanted adverse effects, such as serum sickness disease (SSD), including severe forms (with myocarditis, nephropathies for instance) or other immune complex manifestations such as skin rashes, fever, head ache, arthritis or pseudo meningitis syndrome, etc.

Human ICD have been modeled in animal (F Dixon J Exp Med 1956). The most common and well identified complication in humans following injection of animal IgG is the serum sickness disease (SSD), which is observed in almost 100% of young individual presenting a type 1 diabetes and receiving Thymoglobulin, a purified rabbit IgG anti-T lymphocyte preparation, and in the absence of any other immunosuppressive agents (SE Gitelman et al., The Lancet Diabetes & Endocrinology, 1:306, 2003).

Beside the safety concerns, passive immunotherapy in most of human beings faces the presence of preexisting anti non-human animal Ig which modify the bioavailability of the material injected and possibly its early efficacy. Indeed, it is known that most of human beings already have said anti non-human animal Ig due to their diet and their intestinal biotope. Even in case of an efficient preparation of therapeutic non-human animal Ig and intended to be administered in humans, the severe and highly frequent SSD are thus a major safety and possibly efficacy obstacle for passive immunotherapy in non-immunosuppressed recipients with severe infectious diseases. In addition, the safety concerns may also restrict a wide utilization of preventive campaign in possibly contaminated persons in the patient's vicinity.

What is more, presence of SSD clinical manifestations is a clinical drawback for correctly assessing the result of a therapeutic or preventive procedure using purified polyclonal antibodies. Indeed, clinical symptoms of SSD include notably headache, fever, arthritis or pseudo meningitis syndrome, which can all mislead a correct appraisal of disease evolution.

Accordingly, the inventors have previously demonstrated that antibodies from genetically altered pigs lacking a gene encoding a functional cytidine-5′-monophosphate N-acetyl neuraminic acid hydrolase (CMAH) and a gene encoding a functional α-(1,3)-galactosyltransferase, for producing antibodies (and in particular polyclonal antibodies) directed against a virus for a human, or against at least one molecule derived from said virus, advantageously allow to overcome this drawback.

A method allowing to identify or characterize antibodies according to the present invention falls within the general knowledge of a man skilled in the art.

A method that may be used by the one skilled in the art for identifying or characterizing antibodies according to the invention includes an Enzyme-linked immunosorbent assay (ELISA) wherein, for example, anti-Neu5Gc antibodies and anti-Gal antibodies are used as detection molecules.

In a particular embodiment, a polyclonal antibody of the invention is an immunoglobulin G antibody.

An antibody according to the invention is used in a therapeutically effective amount.

Porcine IgG structure and genetics have been described in literature (Butler et al. 2009-DOI: 10.1007/s00251-009-0356-0). Eleven isotypes called IgG1a, IgG1b, IgG2a, IgG2b, IgG3, IgG4a, IgG4b, IgG5a, IgG5b, IgG6a, IgG6b have been proposed based on analysis of the genomic IgH locus sequences. According to the available knowledge on relative abundance of pig IgG subclasses and Fc domains affinity for Protein A (Butler et al. 2009-DOI: 10.1007/s00251-009-0356-0), during the purification the relative composition of the IgG isotypes in pig DKO anti-RBD polyAb is estimated to be >80% pig IgG1a/b, of 11% IgG2a/b, of 5.5% IgG3, of 3% IgG4a/b, the remaining fraction being other isotypes (IgG5-6). No detectable IgM or IgA isotype is present in pig DKO anti-RBD polyAb.

Polyclonal antibodies of compositions according to the invention may be produced by any technique known in the art, such as, without limitation, any chemical, biological, genetic or enzymatic technique, either alone or in combination.

As it is known in the art, polyclonal antibodies directed against the at least one virus, or at least one molecule derived from the said virus, may be easily obtained by immunizing a pig by administration of an immunogenic composition comprising the target virus or antigens derived thereof (namely molecules derived from said virus), with or without adjuvants.

For illustrative purposes only, polyclonal antibodies used according to the invention can for example be obtained as described in WO2016059161.

Accordingly, an example of method for producing polyclonal antibodies of a composition according to the invention can comprise the steps of:
a) providing a non-genetically altered or a genetically altered pig lacking a first gene selected in a group comprising (i) a gene encoding a functional cytidine-5′-monophosphate N-acetyl neuraminic acid hydrolase (CMAH) and (ii) a gene encoding a functional α-(1,3)-galactosyltransferase (alpha1,3GT, GGTA1 or GT1);
b) immunizing the said pig against the at least one virus, or against at least one molecule derived from the said virus, in particular the spike protein of SARS-COV-2 or a fragment thereof, more particularly at least one molecule selected from the group consisting of the amino acid sequences as set forth in sequences SEQ ID NO: 1 and SEQ ID NO: 2, and is more particularly the amino acid sequence as set forth in sequence SEQ ID NO: 2; and
c) collecting the antibodies contained in a body fluid of the said pig of step b).

Following these steps, the polyclonal antibodies obtained are purified before being used in a composition according to the invention.

This purifying is advantageous in that it notably allows overcoming possible unwanted side effects associated with the presence, within the body fluid, of various cellular contaminants which may involve, by the immunized pig, the formation of corresponding contaminating antibodies.

Said purifying is also advantageous in that it allows obtaining compositions having a desired degree of purity.

Said purifying step falls within the general knowledge of a man skilled in the art. All possible adaptation of any conventional purifying protocol also falls within the general knowledge of a man skilled in the art.

As an appropriate method for obtaining polyclonal antibodies of compositions according to the present invention, may notably be cited the method of fractionated precipitation with ethanol, with ammonium sulfate, with rivanol, with polyethylene glycol or with caprylic acid, the method by passage through ion exchange columns; other methods can involve affinity columns on protein A or G. The antibodies obtained can be then subjected to conventional treatments for their intravenous administration, for example by enzymatic cleavage treatments plasmin, papain or pepsin.

In this regard, may be more particularly cited the protocol implemented in example 3 of EP 0 335 804, which implements an ion exchange chromatography on DEAE cellulose.

Polyclonal antibodies of a composition according to the invention can in particular be able to be present in the body of a human subject to which said composition has been administrated for several weeks days after administration of said composition to the human subject.

Pig polyclonal antibodies of a composition used according to the invention can in particular be directed against a SARS-Cov-2, or against at least one molecule derived from said virus, wherein the said polyclonal antibodies are devoid of a first and a second antigenic determinants being (i)N-glycolylneuraminic acid (Neu5Gc) and (ii) a-1,3-galactose.

In some embodiments, a composition for its use according to the invention is in liquid form.

In some of the embodiments, a composition for its use according to the invention is in a solid form, which includes a lyophilized form.

The composition of the invention may be formulated according to standard methods such as those described in Remington: The Science and Practice of Pharmacy (Lippincott Williams & Wilkins; Twenty first Edition, 2005).

In certain embodiments of a composition according to the invention, said composition may further comprise a pharmaceutical excipient, such as for example one or more charged inorganic carriers. Examples of suitable charged organic carriers include, but are not limited to, saponin, saponin complexes, any one or more components of the immunostimulating complex of saponin, cholesterol and lipid known as ISCOMATRIX.™. (for example the saponin component and/or the phospholipid component), liposomes or oil-in-water emulsions. (The composition and preparation of ISCOMATRIX.™, is described in detail in PCT/SE86/00480, Australian Patent Numbers 558258 and 632067 and EP 0 180 564, the disclosures of which are incorporated herein by reference).

Pharmaceutically acceptable excipients that may be used are for example described in the Handbook of Pharmaceuticals Excipients, American Pharmaceutical Association (Pharmaceutical Press; 6th revised edition, 2009).

Dosages may range from 0.001 to 100 mg or more of polyclonal antibodies as defined herein per kg of body weight (mg/kg) or greater, for example 0.1, 1.0, 10, or 50 mg/kg of body weight, with 1 to 10 mg/kg being preferred. The dosage and frequency of administration may be adapted as detailed below. Dosage and schedule may be different for treatment and prophylaxis usages.

Also, any injection may be followed by any usual procedure to prevent and/or avoid anaphylactic reaction.

Besides, the injection of a composition of the invention can be performed through a large peripheral access or, if possible, through a central catheter.

As is known in the art, adjustments for protein degradation, systemic versus localized delivery, as well as the age, body weight, general health, sex, diet, time of administration, possible allergy, drug interaction and the severity of the condition may be necessary, and is easily determined with routine experimentation by those skilled in the art.

Administration of the composition of the invention may be done in a variety of ways, including, but not limited to, orally, subcutaneously, intravenously, parenterally, intranasally, intrarespiratory (such as nebulization or intra-tracheal spray), intraortically, intraocularly, rectally, vaginally, transdermally, topically (e.g., gels), intraperitoneally, intramuscularly, intrapulmonary or intrathecally.

Administration of the composition of the invention may be done by following the Besredka method.

In a most particular embodiment, a composition according to the invention is in a form suitable for administration by intravenous route.

### 3. Viruses and Associated Diseases

The biological pathogen considered in the present invention may be any virus known for triggering diseases, in particular viruses for which the ADE phenomenon has been observed either naturally or following the administration of a vaccine.

In a particular embodiment, said virus towards a human organism is selected from the group consisting of Coronaviridae family, in particular SARS-COV-2; Dengue virus, Zika virus, Ebola virus, human immunodeficiency virus (HIV), Influenza B virus, hepatitis C virus, Japanese encephalitis virus, Aleutian mink disease parvovirus (AMDV), Human enterovirus 71 (EV71), Ross River virus, Hantavirus, yellow fever virus and a combination thereof.

ADE has been demonstrated or suggested as occurring following infections by, or vaccination against, a plurality of viruses, such as RNA viruses and retroviruses.

In a non-exhaustive manner, ADE has indeed also been demonstrated as occurring following infections by, or vaccination against, Zika virus (Camargos et al., EBioMedicine. 2019 May 23, pii: S2352-3964(19)30315-9); Influenza B virus (Rao et al., Toxicol Sci. 2019 Jun. 1;169(2):409-421); Ebola virus (Furuyama et al., PLOS Pathog. 2016 December 30;12(12):e1006139); HIV (Willey ey al, Retrovirology. 2011 Mar. 14; 8:16); Coxsackievirus (Aswathyraj et al., Microb Pathog. 2018 December;125:7-11); Japanese encephalitis virus (García-Nicolás et al., Viruses. 2017 May 22;9(5)); Aleutian mink disease parvovirus (Kanno et al., J Virol. 1993 December; 67(12):7017-24); hepatitis C virus (Meyer et al., PLOS One. 2011; 6(8):e23699); Human enterovirus 71 (Han et al., Virol J. 2011 Mar. 8; 8:106); Ross River virus (Lidbury and Mahalingam, J Virol. 2000 September;74(18):8376-81); Hantavirus (Yao et al., Arch Virol. 1992; 122(1-2): 107-18); and/or yellow fever virus (Gould and Buckley. J Gen Virol. 1989 June;70 (Pt 6):1605-8).

Macrophage dependent inflammation has further been demonstrated or suggested as occurring following infections by, or vaccination against, a plurality of viruses belonging to the Coronaviridae family.

Accordingly, the present invention also relates to a composition as defined above for its use in the prevention or treatment of diseases caused by macrophage dependent inflammatory cytokine storm in a human subject to, or susceptible to be subjected to, a disease induced by at least one virus, said disease being selected from the group consisting of:
Coronaviridae-related infections, such as SARS, MERS or COVID-19;
Dengue fever, in particular dengue hemorrhagic fever or dengue shock syndrome;
Zika fever disease;
Influenza, in particular influenza due to the Influenza B virus;
Ebola virus disease;
Acquired immunodeficiency syndrome (AIDS);
Encephalitis, in particular Japanese encephalitis;
Aleutian disease;
measles;
hepatitis, in particular hepatitis C;
neurological diseases, and in particular neurological diseases caused by Human enterovirus 71 (EV71);
hand, foot and mouth disease (HFMD);
Ross River fever;
diseases caused by Hantavirus, such as hantavirus hemorrhagic fever with renal syndrome (HFRS) or hantavirus cardiopulmonary syndrome (HCPS); and
yellow fever.

Accordingly, the present invention also relates to a composition as defined above for its use in the prevention or treatment of antibody-dependent macrophage skewing in a human subject to, or susceptible to be subjected to, a disease induced by at least one virus, said disease being selected from the group consisting of Coronaviridae-related infections, such as SARS, MERS or COVID-19.

Furthermore, the invention also relates to a method for preventing and/or treating a disease selected from the group consisting of:
Coronaviridae-related infections, such as SARS, MERS or COVID-19;
Dengue fever, in particular dengue hemorrhagic fever or dengue shock syndrome;
Zika fever disease;
Influenza, in particular influenza due to the Influenza B virus;
Ebola virus disease;
Acquired immunodeficiency syndrome (AIDS);
Encephalitis, in particular Japanese encephalitis;
Aleutian disease;
hepatitis, in particular hepatitis C;
neurological diseases, and in particular neurological diseases caused by Human enterovirus 71 (EV71);
hand, foot and mouth disease (HFMD);
Ross River fever;
diseases caused by Hantavirus, such as hantavirus hemorrhagic fever with renal syndrome (HFRS) or hantavirus cardiopulmonary syndrome (HCPS); and
yellow fever;
comprising administering to a human in need thereof a composition as defined above.

According to some particular embodiments, the present invention also relates to a composition as defined above for its use in the prevention and/or treatment of a disease which is an infection by a RNA virus.

The disease may in particular be an infection by a virus belonging to the Coronaviridae family; in particular selected from the group consisting of: SARS-COV, SARSr-COV WIV1, SARS-COV HKU3, SARS-COV RP3, SARS-COV-2; and their mutants.

### EXAMPLES

### Pig Immunization Strategy

Pig DKO anti-RBD pAb is a polyclonal pig anti-SARS-COV-2 Spike RBD domain glyco-humanized immunoglobulin preparation obtained by immunization of genetically modified pigs with recombinant SARS-COV-2 Spike RBD proteins.

The IgG immunoglobulins purified from the potent serum are used for binding and neutralization assays in following experiment.

CMAH/GGTA1 KO (DKO) pigs were immunized by intramuscular (IM) administrations of SARS-COV-2 RBD spike antigens (SEQ ID NO: 1 and 2). The RBD sequence (SEQ ID NO: 2) was selected based on recent demonstration that it is instrumental in the binding of the spike protein to ACE-2 and that antibodies to RBD consequently inhibit SARS-COV-2 entry into ACE-2-positive cells. RBD antigen was produced in HEK293 cells by conventional methods (Sino Biological-Catalog Number: 40592-V08H). To obtain this antigen, a DNA sequence encoding the SARS-COV-2 (2019-nCOV) Spike Protein (RBD) (YP_009724390.1) (Arg319-Phe541) was expressed with a poly-histidine tag at the C-terminus.

This RBD antigen presents after purification a purity >95% as determined by SDS-PAGE and >95% as determined by SEC-HPLC. The endotoxin content is <1.0 EU per μg protein as determined by the LAL method. The antigen is bioactive, as measured by its binding ability in a functional ELISA. Immobilized human ACE-2 protein can bind SARS-COV-2/2019-nCOV Spike Protein (RBD, His Tag) with an EC50 of 40-80 ng/ml.

### Example 1: Specificity of the Pig DKO Anti-RBD pAb Compared to Irrelevant DKO pAb and pAb from Different Species: Binding Activity by ELISA

The target antigen (SARS-COV-2 (2019-nCOV) Spike S1-mFc protein is immobilized on maxisorp plates at lug/mL in carbonate/bicarbonate buffer is incubated at 4° C. overnight). The plate is washed 3 times in PBS-Tween-0.05%.

The plate is saturated with PBS-Tween-0.05%-2% skimmed milk powder, covered and incubated 2 h at room temperature (RT) and washed 3 times.

Pig DKO anti-RBD pAb or irrelevant DKO pAb (pig IgG) were diluted into PBS-Tween-0.05%-1% skimmed milk powder (between 5 μg/ml and 5 ng/mL) and added into the plate in duplicate, incubated 1 h at RT and washed 3 times.

Pig DKO anti-RBD pAb or irrelevant DKO pAb bind to the target SARS-COV-2 Spike S1-mFc were revealed with secondary HRP-conjugated goat anti-pig IgG Fc antibody diluted in washing buffer, at 1:1000, incubated 1 h at RT and washed 3 times. TMB reagent is added into the plate, incubated up to 15 minutes in the dark and stopped with H2SO4. The plate is read at 450 nm and 630 nm with the TECAN SPARK 10M ELISA reader or equivalent reader.

Following each immunization and after 7 days, blood sample were analysed to measure the polyclonal antibody (pAb) binding activity for the Spike target using the same protocol.

After different immunization courses, end titers in individual animals ranged between 1:100,000 and >1:106. IgG extracted from sera by Protein A chromatography presented an initial EC50 in ELISA of 2.5 μg/mL after two immunization and below 1 μg/mL after 3 or more immunizations.

Binding ELISA results from different DKO Anti-RBD pAb obtained after immunizing at least 5 animals during the immunization campaign showed that polyclonal antibody present an EC50 between 0.05 and 6 μg/ml depending on the pig and the immunization stage.

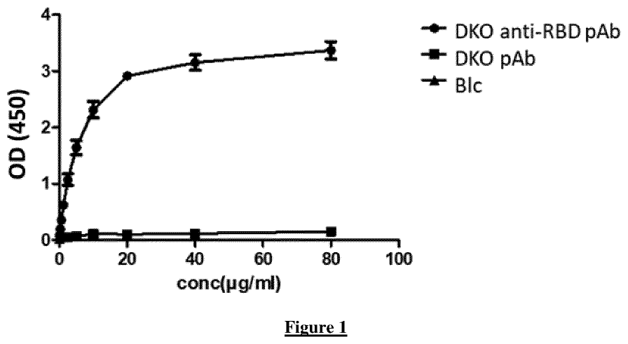

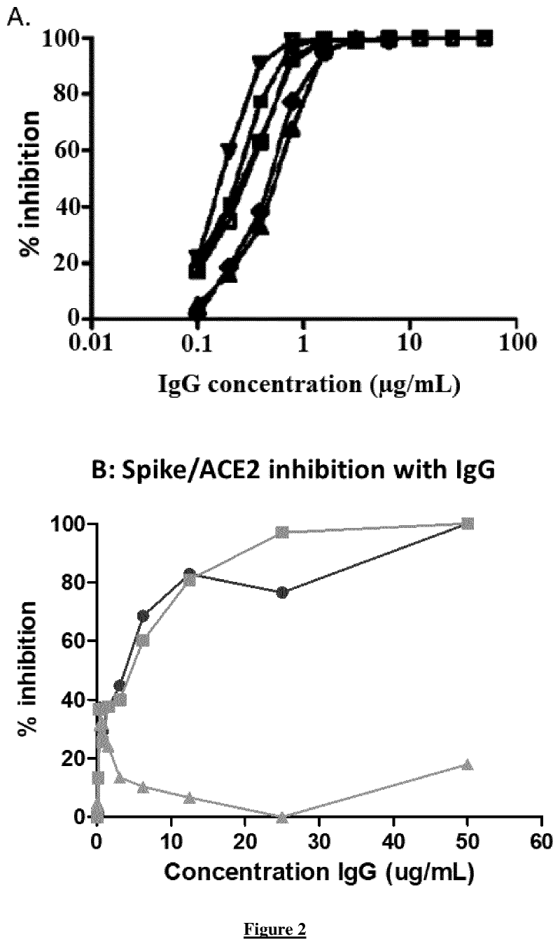

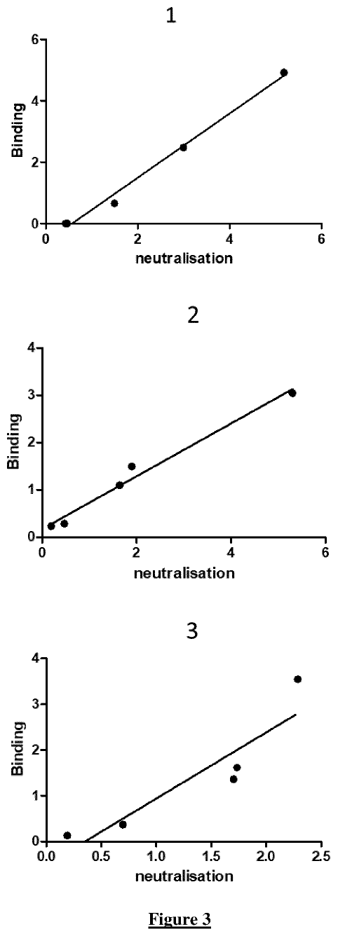

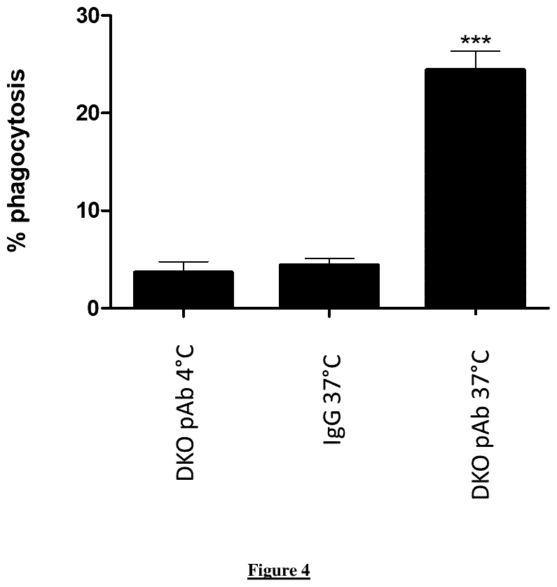

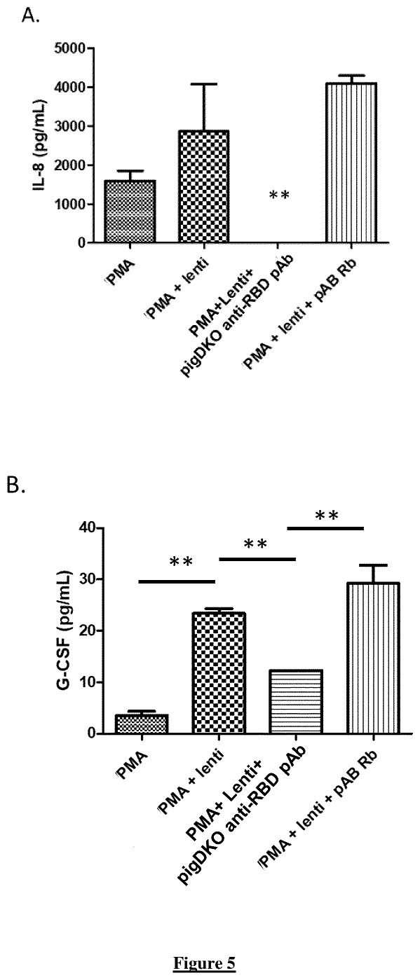

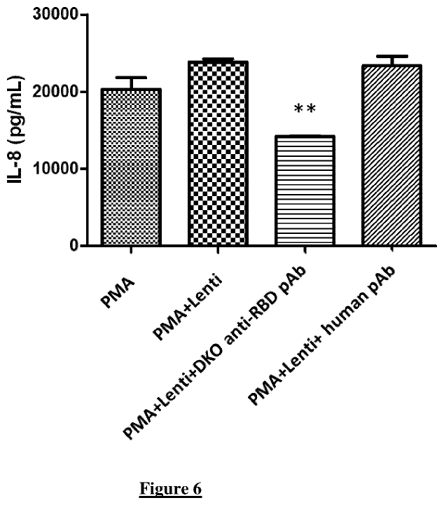

Results shown in FIG. 1 compare the specificity of the binding of the anti-RBD pAb compare to an irrelevant antibody that is not able to bind the spike protein of the SarsCov2 virus.

### Characterization of IgG Composition

It can be estimated from literature data (Butler et al, 2012 http://dx.doi.org/10.1016/j.molimm.2012.07.008) that pig DKO anti-RBD pAb could be composed of >80% pig IgG1a/b, of 11% IgG2a/b, of 5.5% IgG3, of 3% IgG4a/b, the remaining fraction being other isotypes (IgG5-6). Human IgA having a minimal affinity for protein A, it can be expected that pig IgA also minimally binds Protein A. Previous result on other DKO pAb serum purified drug substance revealed nonsignificant IgA content (0,006%). There is also no protein with a size compatible with presence of IgM in the pharmaceutical composition of the invention

### Example 2: Neutralization of SARS-COV-2 by Pig DKO Polyclonal Antibody

In order to analyze the neutralization potency and to confirm data with another independent virus strain, a Cytopathogenic Effect (CPE) assay has been run at VibioSphen, Toulouse, France. The purpose this study was to assess the ability of the serum pool composed of pig DKO anti-RBD pAb to inhibit entry and cytopathic impact of SARS-COV-2 on sensitive cells (Vero E6 Cells). All experimental protocols involving live SARS-COV-2 followed the approved standard operating procedures of the Biosafety Level 3 facility. SARS-COV-2 was isolated from a patient with laboratory-confirmed COVID-19 in Toulouse. The viral isolate was amplified by one additional passage in VeroE6 cells to make working stocks of the virus. Coronavirus Covid-19, Vero E6 Cells (ATCC) were cultured in Dulbecco's modified Eagle's medium (DMEM) supplemented with 10% v/v fetal bovine serum (ATCC), 1% v/v penicillin-streptomycin supplemented with 1% v/v sodium pyruvate. Vero cells were seeded at 1.105 cells per well in 12-well tissue culture plates. At 100% confluence (2 days post-seeding), the cells were washed twice with PBS and six serial dilutions of the virus (1/10 each time) will added to the cells. Following infection with 0.3 ml per well of each dilution, plates will be incubated at 37° C. for 1 h, and the cells were washed with PBS before the addition of 2% w/v agar containing 1 μg ml-5 tosyl phenylalanyl chloromethyl ketone-trypsin (Sigma-Aldrich,) to the cell surface. Plates were left at room temperature for 20-30 min to allow for the overlay to set, and were then incubated at 37° C. for 72 h. Cells will be fixed with 4% v/v paraformaldehyde before both the fixative and agar will be removed and the cells stained with 0.1% w/v Crystal Violet (Fisher) in 20% v/v ethanol. Plaque titres were determined as plaque forming units (p.f.u.) per ml.

CPE reduction assay has been performed as follows: Vero E6 cells were seeded in 96-well clusters at a density of 5000 cells/well 2 day before infection. Two-fold serial dilutions, starting from 200 or 250 μg/mL or from a 1/25 dilution of the serum, where be then mixed with an equal volume of viral solution containing 100 TCID50 of SARS-COV-2. The serum-virus mixture was incubated for 1 hour at 37° C. in a humidified atmosphere with 5% CO2. After incubation, 100 μL of the mixture at each dilution has been added in triplicate to a cell plate containing a semi-confluent VERO E6 monolayer. The plates were then incubated for 3 days at 37° C. in a humidified atmosphere with 5% CO₂.

Cell controls have been infected with Covid-19 at MOI 0,01. The incubation period was 72 h. Uninfected cells have been included as controls to exclude potential cytotoxic/cytostatic effects of the compound treatment. All the samples have been tested in duplicate.

After 3 days of incubation, the plates have been inspected by an inverted optical microscope. The highest serum dilution that prevented formation of plaques (100% efficacy) was taken as the neutralization titre (Table 1).

CPE100 (maximal dilution to reach 100% of neutralization) with pooled hyperimmune sera used to prepare therapeutic IgG reached 1:1600 and the concentration of the IgG preparation (DKO anti-RBDpAb) required to reach CPE100 ranged between 10 and 3.125 μg/mL (Table 1).

TABLE 1
Neutralization activity by Cytopathogenic Effect Assay: neutralizing
titer/concentration of hyperimmune serum and DKO anti-RBD pAb
Cytopathogenic Effect assay run at VibioSphen Serum Pool

| | End Titer r concentration |
|---|---|
| | (μg/ml) to abrogate ECP |
| Sample | (100% efficacy) |
| Serum pool | 1:1600 |
| Batches of different DKO anti-RBDpAb | 3,125 to 10 μg/ml |

Conclusion: Pig hyperimmune glyco-humanized serum and extracted IgG showed neutralizing potency against SARS-COV-2 in vitro, and some extend of cross-neutralization against SARS-COV.

### Example 3: Analysis of the Neutralizing Properties of Pig DKO Anti-RBD pAb Against SARS-COV-2 by ELISA

Neutralizing anti-SARS-COV-2 antibodies have been described as antibodies able to inhibit interaction between SARS-COV-2 spike protein and the SARS-COV-2 receptor ACE-2, according to Rojas et al. (“Convalescent plasma in Covid-19: Possible mechanisms of action”; Autoimmunity Reviews; 2020). Antibodies of the invention have been assessed accordingly.

In an ELISA, a recombinant form of the SARS-COV-2 human receptor ACE2 has been immobilized on plastic. Plates have then been saturated with PBS/5% skimmed milk. Ligand Spike S1 fused with human Fc tag (200 ng/ml) has then added in presence of buffer or dilution of pig serum or dilution of IgG fractions from pig serum. In this configuration, antibodies might compete with Ligand S1 for binding to ACE2. The human Fc tag is then revealed by a specific HRP-conjugated anti-human IgG secondary antibody.

### Results

Pigs immunized with the RBD domain (SEQ ID Nº2) of the SARS-COV-2 spike protein (amino acids 319 to 541 of protein YP_009724390.1, corresponding to SEQ ID Nº1) developed a serum presenting a specific neutralizing end titer of approx. 1:4000. End titer dilution is used here since it is the way human convalescent plasma have been assessed in the literature, leading to the conclusion that an end titer neutralizing titer of 1:40 was sufficient to confer protection, after plasma transfer (as for example in Shen et al.). IgG extracted from sera by Protein A chromatography presented an IC50 comprised between 0,05 μg/ml and 6 μg/ml. After additional immunization, serum presented inhibitory capacities at much higher dilutions and IgG from these sera could be diluted down to <0.1 μg/mL (FIG. 2A).

The data depicted revealed that serum from immunized pigs present neutralizing end titers up approximately to 1:4000. When purified by protein A chromatography, the IgG fraction the inhibition of the preparation turned out to be maximal between 10 and 20 micrograms/mL with an IC50 of approximately 2.5 micrograms/mL (FIG. 2B).

The results provide evidence that the obtained serums on CMAH/GGTA1 KO pigs have a significant neutralizing effect, which is superior to the neutralizing activity observed on convalescent CoVID-19 patients measured to be 40 to 100-fold.

Correlation Between Data from the Neutralization and the Binding ELISA

To characterize the relationship between data from the binding ELISA and the neutralizing activity, a series of pig DKO anti-RBD pAb preparations with different neutralizing activities have been assessed in parallel by binding ELISA. The data shown in FIG. 3 demonstrate that binding is predictive of neutralization activity with a correlation coefficient of 0.97.

### Example 4: Phagocytosis Activity of the Macrophage

Preparation of human monocyte-derived macrophages. Peripheral blood mononuclear cells were isolated by Ficoll-Paque density gradient centrifugation. Monocytes were purified by positive selection using a magnetic cell separation system (Miltenyi Biotec) and were then plated at 2.106 cells/ml in RPMI 1640 medium supplemented with 100 U/ml penicillin, 100 μg/ml streptomycin, 2 mM 1-glutamine and 10% fetal calf serum (FCS). Monocyte differentiation into human Macrophage was carried out for 5-7 days in the presence of hM-CSF (100 ng/ml).

Human lymphocytes were labelled with 1 μM CFSE. CFSE labelled lymphocytes were then incubated with 10 μg/ml IgG (DKO pAb or IgG mAb as control) in RPMI 10% FCS medium for 30 min at 4° C. Lymphocytes were washed twice and cultured with human macrophage (ratio 1:1) in RPMI 10% FCS. After 2-4 hours culture, cells were washed twice and macrophages were labelled with CD14-BV421, for 30 min at 4° C. Cells were washed twice before analysis by flow cytometry. Phagocytosis was assessed as the percentage of double-positive (CFSE+/CD14+) cells. Values were compared by ANOVA followed by the Tukey post hoc test. (*** p<0.001)

FIG. 4 presents results in a percentage of phagocytosis indicating that the phagocytosis is inactive at 4° C. and active at 37° C. when pre activated macrophages are in presence of DKO pAb compare to control IgG. The pig DKO pAb are able to induce the phagocytosis but are unable to induce Antibody Dependent Cell Cytotoxicity (ADCC) by macrophage. These data suggest that pig antibodies could bind human Fc receptor different that those involved in the ADCC.

Example 5: Analysis of the Inflammatory Cytokine Released by Activated Macrophages by ELISA Following Pig Polyclonal Antibodies Treatment in an In Vitro Model of Sars Cov2 Dependent Macrophage Activation

The human myelomonocytic cell line U937 (ATCC CRL-1593.2) or THP1 (ATCC TIB-202) were grown in RPMI 1640 reduced serum medium supplemented with 2 mM L-glutamine, 10% fetal bovine serum (FBS), 5 U/ml penicillin, and 5 mg/ml streptomycin at 37° C. in 5% CO2. For all experiments, the U937 or THP1 cells were differentiated to macrophages by the addition of phorbol 12-myristate 13-acetate (PMA) (Sigma-Aldrich) at a final concentration of 20 ng/ml, during 40 h at 37° C. Then cells were washed twice in RPMI-FBS and seeded in 6 wells-plate (500 000 cells/well). After 24 h hours cells were infected with a lentivirus pseudotyped S1+S2 (p24 titer: 4 ng/ml final) alone or mixed with a polyclonal antibody against SARS-COV2 from different species (Rabbit, pig and human) in 1 mL RPMI medium. All antibodies were added at an equivalent binding activity against the RBD protein. Cells were incubated 2 hours at 37° C. in 5% CO2, then 1 ml of fresh medium was added. Supernantants were collected at 1 and 4 days after infection.

IL-8 and G-CSF cytokines were measured with Elisa kit (IL-8ELISA Kit II-BD OptEIA™-BD Biosciences, G-CSF DuoSet ELISA DY214-05: R&D Systems BD Biosciences) according to manufacturer's instructions.

Results presented FIG. 5 show that at day 4, the preactivated macrophages in presence of Spike pseudotyped lentivirus induce the release of IL8 and G-CSF in the medium characteristic of the macrophage activation. The presence in the culture medium of the DKO anti-RBD pAb inhibit or decrease the release of both cytokines IL8 and G-CSF compare to a pAb from rabbit. Even IL8 secretion is totally inhibited by the U937 cells.

Results presented FIG. 6 show that at day 1, the preactivated macrophages in presence of Spike pseudotyped lentivirus induce the release of IL8 which is partially inhibited when cells are cultured with DKO anti-RBD pAb compare to human polyclonal antibody from Sars Cov2 patient.

Those results suggest that pig DKO anti-RBD pAb are able to down regulate the cytokine storm induced by macrophage and more particularly IL8 secretion which is known as a macrophage cytokine. In this model, pig DKO pAb recognize the spike protein at the lentivirus surface but then is not capable of macrophage activation contrary to the rabbit or human polyclonal antibodies showing that pig DKO polyclonal antibodies are powerful antibodies to down regulate macrophage dependent cytokine storm in several diseases.

SEQUENCE LISTING

SEQ ID No 1
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVEN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRENGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT
SEQ ID No 2
RVQPTESIVRFPNITNLCPFGEVENATRFASVYAWNRKRISNCVADYSV
LYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLS
FELLHAPATVCGPKKSTNLVKNKCVNF

1. A method for preventing or treating a macrophage-dependent inflammation's disease induced by at least one virus wherein said macrophage-dependent inflammation disease is characterized by a cytokine storm in a human that is subject to, or susceptible to be subjected to, the macrophage-dependent inflammation disease, comprising,
administering to the human a pig polyclonal antibody composition comprising,
pig polyclonal antibodies directed against the at least one virus, or against at least one molecule derived from the at least one virus, and
a pharmaceutically acceptable excipient.

2. The method according to claim 1, wherein the pig polyclonal antibodies are devoid of antigenic determinants (i)N-glycolylneuraminic acid (Neu5Gc) and (ii) a-1,3-galactose.

3. The method according to claim 1, wherein the cytokine storm is characterized by an uncontrolled and excessive release of at least one cytokines selected from the group consisting of: Interleukin 8 (IL-8), granulocyte colony-stimulating factor (G-CSF), Interleukin 6 (IL6), TNFalpha, Interleukin 1β (IL1β), MCP-1, CCL-3, CCL-4, CXCL-10 and MIP1alpha and beta.

4. The method according to claim 1, wherein the at least one virus is selected from the group consisting of a Coronaviridae virus, Dengue virus, Zika virus, Ebola virus, human immunodeficiency virus (HIV), Influenza B virus, hepatitis C virus, Japanese encephalitis virus, Aleutian mink disease parvovirus (AMDV), Human enterovirus 71 (EV71), Ross River virus, Hantavirus, yellow fever virus and a combination thereof.

5. The method according to claim 1, wherein the pig polyclonal antibodies of the composition are Immunoglobulin G.

6. The method according to any claim 1, wherein the macrophage-dependent inflammation disease induced by the at least one virus is an infection.

7. The method according to claim 1, wherein the macrophage-dependent inflammation disease is an infection by an RNA virus.

8. The method according to claim 1, wherein the macrophage-dependent inflammation disease is an infection by a virus belonging to the Coronaviridae family.

9. The method according to claim 1, wherein the macrophage-dependent inflammation disease is Hemophagocytic lymphohistiocytosis (HLH).

10. The method according to claim 1, wherein the pig polyclonal antibodies of the composition have a binding activity by ELISA to the at least one virus or to the at least one molecule derived from the at least one virus between 0.05 μg/mL and 6 μg/mL.

11. The method according to claim 10, wherein the at least one molecule derived from the at least one virus is the spike protein of SARS-COV-2 or a fragment thereof.

12. The method according to claim 1, wherein the pig polyclonal antibodies of the composition have a neutralization activity by ELISA to the at least one virus or to the at least one molecule derived from the at least one virus of between 0.10 μg/mL and 11 μg/mL.

13. The method according to claim 12, wherein the at least one molecule derived from the at least one virus is the spike protein of SARS-COV-2 or a fragment thereof.

14. The method according to claim 3, wherein said method reduces the uncontrolled and excessive release of at least one cytokine, in the human by at least 10%, 20%, 30%, 40%, 50%, 60%, 70%, 80%, 90% or 95%, as compared to the uncontrolled and excessive release of the at least one cytokine in a human to whom the compositions is not administered.

15. The method of claim 4, wherein the Coronaviridae virus is a SARS-CoV-2 virus.
